(19) United States
(12) Patent Application Publication
(10) Pub. No.: US 2024/0148954 A1
(43) Pub. Date: May 9, 2024

(54) ASPIRATION REFLUX CONTROL SYSTEM

(71) Applicant: JOHNSON & JOHNSON SURGICAL VISION, INC., Irvine, CA (US)

(72) Inventors: Vadim Gliner, Haifa (IL); Assaf Govari, Haifa (IL); Adam Walter Toner, Ladera Ranch, CA (US)

(21) Appl. No.: 17/980,689

(22) Filed: Nov. 4, 2022

(51) Int. Cl. A61M 1/00; A61F 9/007

(52) U.S. Cl. CPC A61M 1/772; A61F 9/00781; A61B 2217/005; A61B 2217/007; A61M 2205/078; A61M 2205/3334; A61M 2205/502; A61M 2210/0612

(57) ABSTRACT

In one exemplary mode, a phacoemulsification system includes a phacoemulsification probe to be inserted into an eye, an irrigation line to provide irrigation fluid into the eye, an aspiration line to convey aspiration fluid from the eye, an aspiration pump to pump the aspiration fluid from the eye, a pump controller to control a flow direction and rate of the aspiration pump, and an aspiration rate user input device to provide a signal indicative of user actuation of the aspiration rate user input device, wherein the pump controller is configured to receive the signal provided by the aspiration rate user input device, and reverse the flow direction and set the flow rate of the aspiration pump at which to pump the aspiration fluid into the eye, the flow rate in the reverse flow direction being set responsively to an actuation rate at which the aspiration rate user input device is actuated.

### FIELD OF THE DISCLOSURE

The present disclosure relates to phacoemulsification systems, and in particular, but not exclusively to, reflux control in phacoemulsification systems.

### BACKGROUND

A cataract is a clouding and hardening of the eye's natural lens, a structure which is positioned behind the cornea, iris, and pupil. The lens is mostly made up of water and protein and as people age these proteins change and may begin to clump together obscuring portions of the lens. To correct this a physician may recommend phacoemulsification cataract surgery. Before the procedure, the surgeon numbs the area with anesthesia. Then a small incision is made in the sclera or clear cornea of the eye. Fluids are injected into this incision to support the surrounding structures. The anterior surface of the lens capsule is then removed to gain access to the cataract. The surgeon then uses a phacoemulsification probe, which has an ultrasonic handpiece with a titanium or steel needle. The tip of the needle vibrates at ultrasonic frequency to sculpt and emulsify the cataract while a pump aspirates lens particles and fluid from the eye through the tip. The pump is typically controlled with a microprocessor.

Any suitable pump may be used, for example, a peristaltic and/or a venturi type of pump. Aspirated fluids are replaced with irrigation of a balanced salt solution to maintain the anterior chamber of the eye. After removing the cataract with phacoemulsification, the softer outer lens cortex is removed with suction. An intraocular lens (IOL) is introduced into the empty lens capsule. Small struts called haptics hold the IOL in place. Once correctly installed the IOL restores the patient's vision.

### BRIEF DESCRIPTION OF THE DRAWINGS

The present disclosure will be understood from the following detailed description, taken in conjunction with the drawings in which:

FIG. 1 is a schematic pictorial illustration of an ophthalmic surgical system constructed and operative in accordance with an exemplary mode of the present disclosure;

FIG. 2 is a block diagram view of part of the system of FIG. 1; and

FIG. 3 is a flowchart including steps in a method of operation of the system of FIG. 1.

### DESCRIPTION OF EXAMPLES

### Overview

During a phacoemulsification procedure, the physician uses a vacuum created by an aspiration pump used to grab suitable material, e.g., a lens capsule, or to remove cataract debris from the eye. In some cases, the physician may inadvertently capture some anatomical structure, e.g., the posterior capsule, which could result in damage to the eye. When this happens, the physician tends to suddenly release the pedal (which actuates the aspiration pump). The release of the pedal automatically activates reflux mode in which the aspiration pump flow is reversed thereby leading to release of the captured anatomical structure. However, the same reflux mode is applied regardless of how quickly or slowly the physician releases the pedal. This therefore leads to excess fluid in the eye irrespective of whether reflux is needed or not, or whether different levels of reflux are needed. Excess fluid in the eye may lead to excess intraocular pressure which may be dangerous to the eye.

Exemplary modes of the present disclosure solve at least some of the above drawbacks by performing reflux according to the rate at which the physician releases the pedal (or other actuator) as the rate at which the physician releases the pedal is generally indicative of the severity of the inadvertent capture. In cases where the physician releases the pedal slowly, the reflux mode may not be required at all.

In some exemplary modes, a phacoemulsification system monitors the rate (e.g., velocity and/or acceleration) at which the physician releases the pedal (or another actuator) and adjusts the rate of reflux based on the monitored rate. In some exemplary modes, the aspiration pump may be controlled using a proportional-integral-derivative (PID) controller with suitable control parameters. The PID controller may increase the reflux rate for instances of fast release of the pedal (or another actuator) and decrease the reflux rate for instances of slow release of the pedal (or another actuator). The control parameters are dynamically adjusted based on the rate at which the pedal is released. In some exemplary modes, when the rate of release of the pedal (or other actuator) is below a defined threshold, reflux may be deactivated.

### System Description

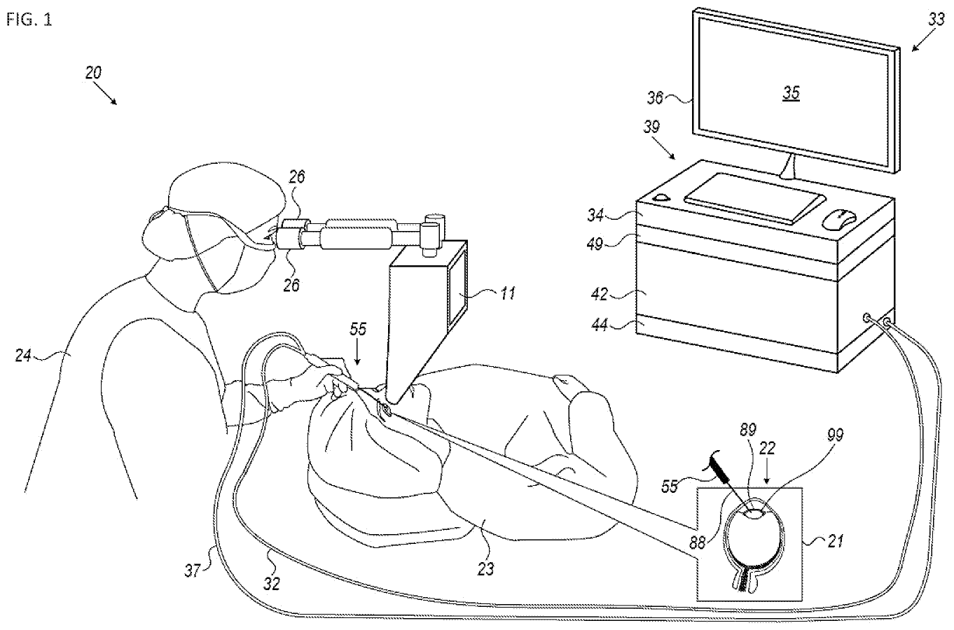

Reference is now made to FIG. 1, which is a schematic pictorial illustration of an ophthalmic surgical system 20, in accordance with an exemplary mode of the present disclosure. System 20 is configured to carry out various types of ophthalmic procedures, such as cataract surgery.

In some exemplary modes, system 20 comprises a medical instrument, in the present example a phacoemulsification handpiece, also referred to herein as a tool 55, used by a surgeon 24 to carry out the cataract surgery. In other exemplary modes, system 20 may comprise other surgical tools, such as but not limited to an irrigation and aspiration (I/A) handpiece, a diathermy handpiece, a vitrectomy handpiece, and similar instruments.

Reference is now made to an inset 21 showing a sectional view of the surgical procedure carried out in an eye 22 of a patient 23. In some exemplary modes, surgeon 24 applies tool 55 for treating eye 22, and in the present example, surgeon 24 inserts a needle 88 of tool 55 into eye 22. In the example of inset 21, during a cataract surgical procedure, surgeon 24 inserts needle 88 into a capsular bag 89 so as to emulsify a lens 99 of eye 22.

Reference is now made back to the general view of FIG. 1. In some exemplary modes, system 20 comprises a console 33, which comprises a processor 34, a memory 49, a generator 44 and a cartridge 42. In some exemplary modes, cartridge 42 comprises pumping sub-systems (not shown) configured to apply, via multiple tubes 32, irrigation fluids (not shown) into eye 22 and to draw eye fluids away from eye 22 into cartridge 42. In the context of the present disclosure, the term “eye fluid” refers to any mixture of natural eye fluid, irrigation fluid and lens material. Note that tubes 32 may comprise an irrigation tube for supplying the irrigation fluid into eye 22, and a separate aspiration tube for drawing the eye fluids away from eye 22.

In some exemplary modes, generator 44 is electrically connected to tool 55, via a plurality of wires referred to herein as an electrical cable 37. Generator 44 is configured to generate one or more voltage periodic (e.g., sinusoidal) signals, also referred to herein as periodic signals, having one or more frequencies, respectively. Generator 44 is further configured to generate a plurality of driving signals, so as to vibrate needle 88 of tool 55 in accordance with a predefined pattern, so as to emulsify lens 99 of eye 22.

In some exemplary modes, processor 34 typically comprises a general-purpose computer, with suitable front end and interface circuits for controlling generator 44, cartridge 42 and other components of system 20.

In practice, some or all of the functions of the processor 34 may be combined in a single physical component or, alternatively, implemented using multiple physical components. These physical components may comprise hard-wired or programmable devices, or a combination of the two. In some exemplary modes, at least some of the functions of the processor 34 may be carried out by a programmable processor under the control of suitable software. This software may be downloaded to a device in electronic form, over a network, for example. Alternatively, or additionally, the software may be stored in tangible, non-transitory computer-readable storage media, such as optical, magnetic, or electronic memory.

In some exemplary modes, system 20 comprises an ophthalmic surgical microscope 11, such as ZEISS OPMI LUMERA series or ZEISS ARTEVO series supplied by Carl Zeiss Meditec AG (Oberkochen, Germany), or any other suitable type of ophthalmic surgical microscope provided by other suppliers. Ophthalmic surgical microscope 11 is configured to produce stereoscopic optical images and two-dimensional (2D) optical images of eye 22. During the cataract surgery, surgeon 24 typically looks though eyepieces 26 of ophthalmic surgical microscope 11 for viewing eye 22.

In some exemplary modes, console 33 comprises a display 36 and input devices 39, which may be used by surgeon 24 for controlling tool 55 and other components of system 20. Moreover, processor 34 is configured to display on display 36, an image 35 received from any suitable medical imaging system for assisting surgeon to carry out the cataract surgery.

This particular configuration of system 20 is shown by way of example, in order to illustrate certain problems that are addressed by exemplary modes of the present disclosure and to demonstrate the application of these exemplary modes in enhancing the performance of such a system. Exemplary modes of the present disclosure, however, are by no means limited to this specific sort of example system, and the principles described herein may similarly be applied to other sorts of ophthalmic and other minimally invasive and surgical systems.

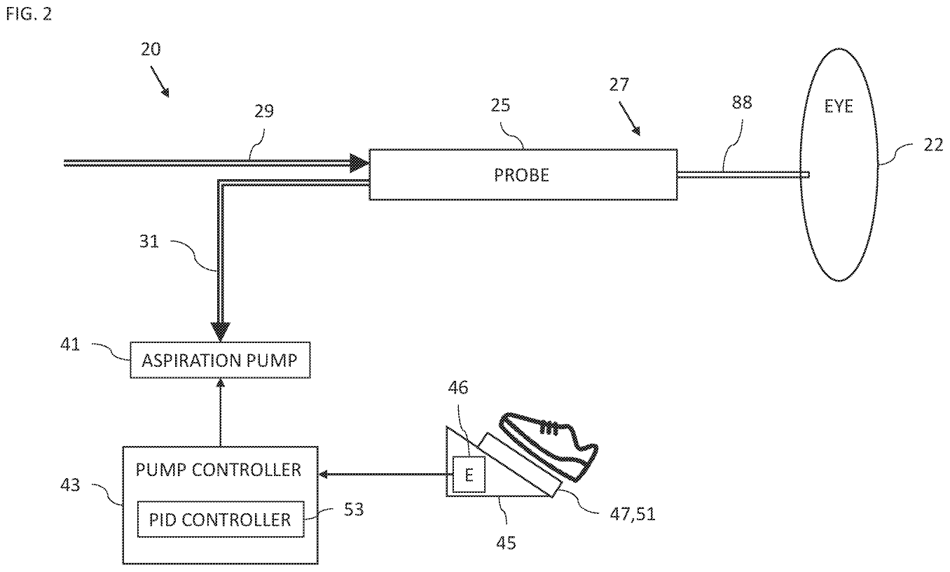

Reference is now made to FIG. 2, which is a block diagram view of part of the system 20 of FIG. 1. The tool 55 includes a phacoemulsification probe 25 having a distal end 27 comprising the needle 88, and configured to be inserted into the eye 22. The system 20 includes an irrigation line 29 configured to be connected to the probe 25 and to provide irrigation fluid into the eye 22. The phacoemulsification probe 25 also includes an irrigation channel (not shown) to carry irrigation fluid from the irrigation line 29 to the distal end 27. The system 20 also includes an aspiration line 31 configured to be connected to the probe 25 and to convey aspiration fluid from the eye 22. The phacoemulsification probe 25 also includes an aspiration channel (not shown) to carry aspiration fluid from the eye 22 to the aspiration line 31. The aspiration channel extends from the distal end of the needle 88 to the proximal end of the phacoemulsification probe 25. The system 20 includes an aspiration pump 41 configured to be connected to the aspiration line 31 and to pump the aspiration fluid from the eye 22.

The system 20 includes a pump controller 43 configured to control a flow direction and a flow rate of the aspiration pump 41. In some exemplary modes, the pump controller 43 includes a proportional-integral-derivative controller 53, which is configured to control the flow rate in the reverse or forward flow direction responsively to control parameters.

The system 20 also includes an aspiration rate user input device 45 configured to provide a signal indicative of user actuation of the aspiration rate user input device 45. In some exemplary modes, the aspiration rate user input device 45 includes a widget 47, which is actuated by the surgeon 24 to adjust the flow rate of the aspiration pump 41. An actuation rate of the aspiration rate user input device 45 is used by the pump controller 43 to control the flow direction and flow rate of the aspiration pump 41 as described in more detail below. The actuation rate is computed from the signal provided by the aspiration rate user input device 45. The computation of the actuation rate from the signal may depend on the type of sensor included in the aspiration rate user input device 45. For example, the aspiration rate user input device 45 may include one or more of the following: a gyroscope, a variable resistor, and/or a strain gauge, by way of example.

In some exemplary modes, the actuation rate at which the aspiration rate user input device 45 is actuated is a rate of change of position and/or velocity of the widget 47. In some exemplary modes, the widget 47 includes a foot pedal 51, and the foot pedal 51 is adjusted, e.g., released or depressed, to reduce or increase the aspiration flow rate.

In some exemplary modes, the rate of change of the position and/or velocity of the foot pedal 51 is based on the rate of change of release of the foot pedal (e.g., when the foot pedal is released to reduce the aspiration flow rate). In other exemplary modes, the rate of change of the position and/or velocity of the foot pedal is based on the rate of change of pressing of the foot pedal (e.g., when the foot pedal is depressed (e.g., backwards) to reduce the aspiration flow rate).

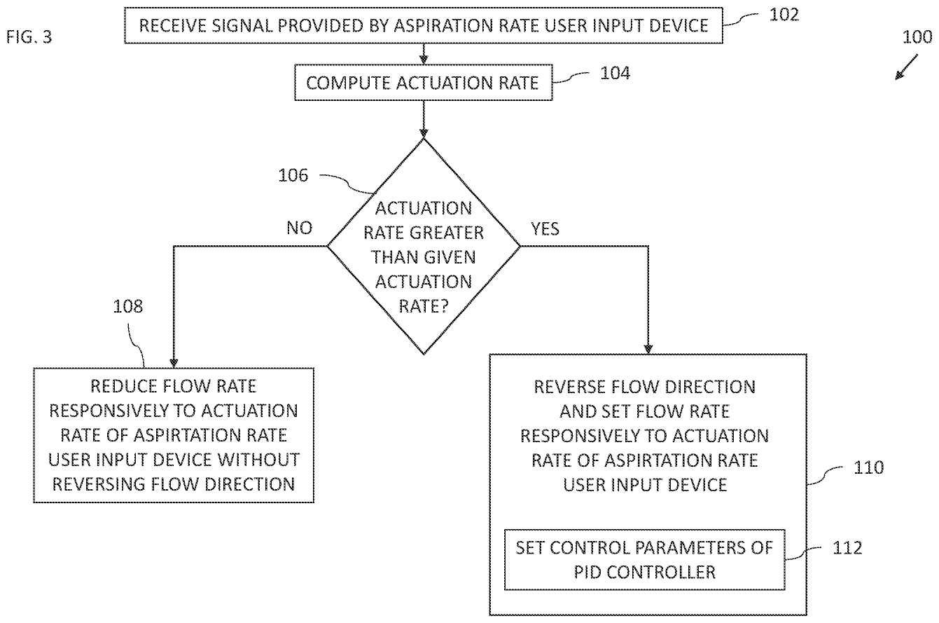

Reference is now made to FIG. 3, which is a flowchart 100 including steps in a method of operation of the system 20 of FIG. 1. The pump controller 43 is configured to receive the signal provided by the aspiration rate user input device 45 (block 102), for example, based on actuation of the foot pedal 51. The pump controller 43 is configured to compute the actuation rate of the aspiration rate user input device 45 (block 104) based on the received signal. At a decision block 106, the pump controller 43 is configured to determine if the computed actuation rate is greater than a given actuation rate. If the computed actuation rate is less than the given actuation rate, the pump controller 43 is configured to reduce the flow rate of the aspiration pump 41 at which to pump the aspiration fluid from the eye 22, without reversing the flow direction (block 108). If the computed actuation rate is greater than the given actuation rate, the pump controller 43 is configured to reverse the flow direction of the aspiration fluid and set the value of the flow rate in the reverse flow direction responsively to the computed actuation rate at which the aspiration rate user input device is actuated (block 110). For example, the flow rate in the reverse direction is set as a function of the actuation rate so that the flow rate is higher if the actuation is higher, and vice-versa. In some exemplary modes, the pump controller 43 is configured to set the control parameters of the proportional-integral-derivative controller 53 responsively to the computed actuation rate at which the aspiration rate user input device 45 is actuated. Therefore, the proportional-integral-derivative controller 53 is configured to control the flow rate of the aspiration pump 41 in the reverse flow direction responsively to the control parameters. The control parameters may include a proportional parameter (Kp), an integral parameter (Ki) and a derivative parameter (Kd). The control parameters may be set such that Kp is a function of dP/dt (the rate of change of P) where P is the displacement of the pedal and dP/dt is how fast the pedal is released. In some embodiments, the other parameters Ki and/or Kd may (also) be set as a function of dP/dt. In one example, where dP/dt is in the range of 0-255, Kp may be set to 2/255×dP/dt, Ki may be set to 0.6, and Kd may be set to 5. The value dP/dt may be equal to, or derived from, the actuation rate described above. The aspiration rate user input device 45 may include an encoder 46, which provides the actuation rate. For example, the encoder 46 may provide a signal indicative of the actuation rate. In some example embodiments, aspiration rate user input device 45 is a foot pedal that is operated by the physician to activate or deactivate the aspiration flow. Optionally, the foot pedal includes encoder 46 that monitors position of the foot pedal. In some example embodiments, output from the encoder is used to determine a position of the foot pedal as well as to compute a rate at which the physician releases the foot pedal. Typically, the foot pedal is configured to allow the physician to control the irrigation, aspiration as well as the ultrasound energy provided by the handpiece.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 72% to 108%.

### EXAMPLES

Example 1: A phacoemulsification system, comprising: a phacoemulsification probe having a distal end comprising a needle, and configured to be inserted into an eye; an irrigation line configured to be connected to the probe and to provide irrigation fluid into the eye; an aspiration line configured to be connected to the probe and to convey aspiration fluid from the eye; an aspiration pump configured to be connected to the aspiration line and to pump the aspiration fluid from the eye; a pump controller configured to control a flow direction and a flow rate of the aspiration pump; and an aspiration rate user input device configured to provide a signal indicative of user actuation of the aspiration rate user input device, wherein: the pump controller is configured to: receive the signal provided by the aspiration rate user input device; and reverse the flow direction and set the flow rate of the aspiration pump at which to pump the aspiration fluid into the eye, the flow rate in the reverse flow direction being set responsively to an actuation rate at which the aspiration rate user input device is actuated.

Example 2: The system according to example 1, wherein: the aspiration rate user input device includes a widget; and the actuation rate at which the aspiration rate user input device is actuated is a rate of change of position of the widget.

Example 3: The system according to example 2, wherein the widget includes a foot pedal, and the rate of change of position of the widget is a rate of change of release of the foot pedal.

Example 4: The system according to example 2, wherein the widget includes a foot pedal, and the rate of change of position of the widget is a rate of change of pressing of the foot pedal.

Example 5: The system according to example 1, wherein: the aspiration rate user input device includes a widget; and the actuation rate at which the aspiration rate user input device is actuated is a rate of change of velocity of the widget.

Example 6: The system according to example 5, wherein the widget includes a foot pedal, and the rate of change of velocity of the widget is a rate of change of release of the foot pedal.

Example 7: The system according to example 5, wherein the widget includes a foot pedal, and the rate of change of velocity of the widget is a rate of change of pressing of the foot pedal.

Example 8: The system according to example 1, wherein the pump controller includes a proportional integral derivative (PID) controller configured to control the flow rate in the reverse flow direction responsively to control parameters, wherein the pump controller is configured to set the control parameters responsively to the actuation rate at which the aspiration rate user input device is actuated.

Example 9: The system according to example 1, wherein the pump controller is configured to set the flow rate in the reverse flow direction responsively to the actuation rate at which the aspiration rate user input device is actuated being greater than a given actuation rate.

Example 10: The system according to example 9, wherein the pump controller is configured to reduce the flow rate of the aspiration pump at which to pump the aspiration fluid from the eye, without reversing the flow direction responsively to the actuation rate at which the aspiration rate user input device is actuated being less than a given actuation rate.

Example 11: A phacoemulsification method, comprising: inserting a phacoemulsification probe having a distal end comprising a needle into an eye; providing irrigation fluid into the eye via an irrigation line connected to the probe; pumping aspiration fluid from the eye via an aspiration line connected to the probe; controlling a flow direction and a flow rate of the pumping; providing a signal indicative of user actuation of an aspiration rate user input device; receiving the signal provided by the aspiration rate user input device; and reversing the flow direction and setting the flow rate of the pumping at which to pump the aspiration fluid into the eye, the flow rate in the reverse flow direction being set responsively to an actuation rate at which the aspiration rate user input device is actuated.

Example 12: The method according to example 11, wherein: the aspiration rate user input device includes a widget; and the actuation rate at which the aspiration rate user input device is actuated is a rate of change of position of the widget.

Example 13: The method according to example 12, wherein the widget includes a foot pedal, and the rate of change of position of the widget is a rate of change of release of the foot pedal.

Example 14: The method according to example 12, wherein the widget includes a foot pedal, and the rate of change of position of the widget is a rate of change of pressing of the foot pedal.

Example 15: The method according to example 11, wherein: the aspiration rate user input device includes a widget; and the actuation rate at which the aspiration rate user input device is actuated is a rate of change of velocity of the widget.

Example 16: The method according to example 15, wherein the widget includes a foot pedal, and the rate of change of velocity of the widget is a rate of change of release of the foot pedal.

Example 17: The method according to example 15, wherein the widget includes a foot pedal, and the rate of change of velocity of the widget is a rate of change of pressing of the foot pedal.

Example 18: The method according to example 11, further comprising setting control parameters of a proportional integral derivative (PID) controller to control the flow rate in the reverse flow direction responsively to the actuation rate at which the aspiration rate user input device is actuated.

Example 19: The method according to example 11, wherein the setting includes setting the flow rate in the reverse flow direction responsively to the actuation rate at which the aspiration rate user input device is actuated being greater than a given actuation rate.

Example 20: The method according to example 19, further comprising reducing the flow rate of the pumping at which to pump the aspiration fluid from the eye, without reversing the flow direction, responsively to the actuation rate at which the aspiration rate user input device is actuated being less than a given actuation rate.

Various features of the disclosure which are, for clarity, described in the contexts of separate examples may also be provided in combination in a single example. Conversely, various features of the disclosure which are, for brevity, described in the context of a single example may also be provided separately or in any suitable sub-combination.

The examples described above are cited by way of example, and the present disclosure is not limited by what has been particularly shown and described hereinabove. Rather the scope of the disclosure includes both combinations and sub-combinations of the various features described hereinabove, as well as variations and modifications thereof which would occur to persons skilled in the art upon reading the foregoing description and which are not disclosed in the prior art.

What is claimed is:

1. A phacoemulsification system, comprising:
a phacoemulsification probe having a distal end comprising a needle, and configured to be inserted into an eye;
an irrigation line configured to be connected to the probe and to provide irrigation fluid into the eye;
an aspiration line configured to be connected to the probe and to convey aspiration fluid from the eye;
an aspiration pump configured to be connected to the aspiration line and to pump the aspiration fluid from the eye;
a pump controller configured to control a flow direction and a flow rate of the aspiration pump; and
an aspiration rate user input device configured to provide a signal indicative of user actuation of the aspiration rate user input device, wherein:
the pump controller is configured to: receive the signal provided by the aspiration rate user input device; and reverse the flow direction and set the flow rate of the aspiration pump at which to pump the aspiration fluid into the eye, the flow rate in the reverse flow direction being set responsively to an actuation rate at which the aspiration rate user input device is actuated.

2. The system according to claim 1, wherein:
the aspiration rate user input device includes a widget; and
the actuation rate at which the aspiration rate user input device is actuated is a rate of change of position of the widget.

3. The system according to claim 2, wherein the widget includes a foot pedal, and the rate of change of position of the widget is a rate of change of release of the foot pedal.

4. The system according to claim 2, wherein the widget includes a foot pedal, and the rate of change of position of the widget is a rate of change of pressing of the foot pedal.

5. The system according to claim 1, wherein:
the aspiration rate user input device includes a widget; and
the actuation rate at which the aspiration rate user input device is actuated is a rate of change of velocity of the widget.

6. The system according to claim 5, wherein the widget includes a foot pedal, and the rate of change of velocity of the widget is a rate of change of release of the foot pedal.

7. The system according to claim 5, wherein the widget includes a foot pedal, and the rate of change of velocity of the widget is a rate of change of pressing of the foot pedal.

8. The system according to claim 1, wherein the pump controller includes a proportional integral derivative (PID) controller configured to control the flow rate in the reverse flow direction responsively to control parameters, wherein the pump controller is configured to set the control parameters responsively to the actuation rate at which the aspiration rate user input device is actuated.

9. The system according to claim 1, wherein the pump controller is configured to set the flow rate in the reverse flow direction responsively to the actuation rate at which the aspiration rate user input device is actuated being greater than a given actuation rate.

10. The system according to claim 9, wherein the pump controller is configured to reduce the flow rate of the aspiration pump at which to pump the aspiration fluid from the eye, without reversing the flow direction responsively to the actuation rate at which the aspiration rate user input device is actuated being less than a given actuation rate.

11. A phacoemulsification method, comprising:
inserting a phacoemulsification probe having a distal end comprising a needle into an eye;
providing irrigation fluid into the eye via an irrigation line connected to the probe;
pumping aspiration fluid from the eye via an aspiration line connected to the probe;
controlling a flow direction and a flow rate of the pumping;
providing a signal indicative of user actuation of an aspiration rate user input device;
receiving the signal provided by the aspiration rate user input device; and
reversing the flow direction and setting the flow rate of the pumping at which to pump the aspiration fluid into the eye, the flow rate in the reverse flow direction being set responsively to an actuation rate at which the aspiration rate user input device is actuated.

12. The method according to claim 11, wherein:
the aspiration rate user input device includes a widget; and
the actuation rate at which the aspiration rate user input device is actuated is a rate of change of position of the widget.

13. The method according to claim 12, wherein the widget includes a foot pedal, and the rate of change of position of the widget is a rate of change of release of the foot pedal.

14. The method according to claim 12, wherein the widget includes a foot pedal, and the rate of change of position of the widget is a rate of change of pressing of the foot pedal.

15. The method according to claim 11, wherein:
the aspiration rate user input device includes a widget; and
the actuation rate at which the aspiration rate user input device is actuated is a rate of change of velocity of the widget.

16. The method according to claim 15, wherein the widget includes a foot pedal, and the rate of change of velocity of the widget is a rate of change of release of the foot pedal.

17. The method according to claim 15, wherein the widget includes a foot pedal, and the rate of change of velocity of the widget is a rate of change of pressing of the foot pedal.

18. The method according to claim 11, further comprising setting control parameters of a proportional integral derivative (PID) controller to control the flow rate in the reverse flow direction responsively to the actuation rate at which the aspiration rate user input device is actuated.

19. The method according to claim 11, wherein the setting includes setting the flow rate in the reverse flow direction responsively to the actuation rate at which the aspiration rate user input device is actuated being greater than a given actuation rate.

20. The method according to claim 19, further comprising reducing the flow rate of the pumping at which to pump the aspiration fluid from the eye, without reversing the flow direction, responsively to the actuation rate at which the aspiration rate user input device is actuated being less than a given actuation rate.
